(19) United States
(12) Patent Application Publication
(10) Pub. No.: US 2017/0112189 A1
(43) Pub. Date: Apr. 27, 2017

(54) Multi Purpose Self Rolled and Paperless Smoking Apparatus for any kind of Tobacco

(71) Applicant: Eli Stern, San Diego, CA (US)

(72) Inventor: Eli Stern, San Diego, CA (US)

(21) Appl. No.: 15/334,180

(22) Filed: Oct. 25, 2016

Related U.S. Application Data
(60) Provisional application No. 62/246,070, filed on Oct. 25, 2015.

(51) Int. Cl. A24F 13/06; A24F 1/32; A61K 36/185; A24F 1/28

(52) U.S. Cl. CPC A24F 13/06; A24F 1/28; A24F 1/32; A61K 36/185

(57) ABSTRACT

This invention has multiple uses for smoking self-rolled cigarettes or the bigger ones called blunts made out of cigars or cigar rolling papers and even work as a paperless cigarette, paperless joint, or paperless joint. This embodiment has 4 parts of aluminum other metals or heat resistant materials may be used and 2 metal screens. A blunt or joint may be placed into the device to be used as a holder tube 2 that is cone shaped for adjustable fitting for joints, or smaller roller cigarettes or into tube 3 that is cone shaped for adjustable fitting for blunts, or larger roller cigarettes that protects the user from getting tobacco or legal marijuana into their mouth, it may also be used as a paperless joint, cigarette or blunt, by putting the tobacco or legal marijuana direct into the device in tube (3). It can also be used to store an unfinished hand rolled or paperless rolled cigarette, legal joint or blunt. It also supply a better smoke like one would get from a water pipe because of its built in filter with screen in tube (1).

### BACKGROUND

When smoking a self-rolled Cigarette whether small from a regular rolling paper or larger with a cigar paper, you get tobacco, or now in some states legal marijuana in your mouth. The intake from a self rolled cigarette when your mouth is directly on the cigarette is not as good as when smoking from a water pipe or a regular pipe. You also have to roll it in either a thin paper or cigar paper. This device which can hold both a thin self-rolled cigarette and a self-rolled cigarette rolled in a cigar paper, as well as it can be used as the paper itself and it contains screens to keep the tobacco or marijuana from entering your mouth. You get a better smoother hit and no tobacco in your mouth works as a rolled cigarette even without cigarette marijuana or cigar paper .It also works as a holder for self-rolled cigarettes whether paperless, or a self-rolled cigarette rolled in a cigar paper or marijuana papers.

### SUMMARY OF THE INVENTION

A Device for smoking self-rolled tobacco with many uses and many benefits to the smoker regardless of the size of the self-rolled cigarette it works with different size rolling papers which you stick in the device, as well as paperless you may put the tobacco right into the device and smoke it with the need for papers. The device has 2 filters which keeps the tobacco from the self-rolled as well as paperless if you use the device in that fashion out of your mouth. The built in filter also allows a better way to inhale more of the tobacco as more air intake acts as like a small water pipe giving a cleaner smoother inhalation and a larger inhale of the tobacco. Compared to smoking directly out of a self-rolled cigarette or cigar.

### DESCRIPTION OF THE DRAWINGS

FIG. 1A. FIG. 1 shows a perspective view of the device number (1) is the Filter number (2) is the smaller piece number (3) is the larger piece number (4) is the cap

FIG. 1B. FIG. 1 shows a Orthographic view of the device number (1) is the Filter number (2) is the smaller piece number (3) is the larger piece number (4) is the cap

FIG. 2. FIG. 2 shows the device separated by pieces in a top view with the screens attached to the Filter in number (1) and the Cap in number (4) number (2) is the smaller for smaller piece for self-rolled tobacco cigarettes number (3) is for larger self-rolled Cigarettes such as when using Cigar paper.

FIG. 3. Shows the device in exploited view separated by pieces number (5) shows the filter and its threads number (6) shows the heat dissipating fin that also acts as the inward thread. Number (7) shows the threads on the thinner rolled tobacco piece (8) shows the heat dissipating fin that also acts as the inward thread. (9) Shows the fin on the thicker rolled tobacco piece number (10) shows the threads on same thicker tobacco peace. number (11) Shows the threads on the cap number (12) show the end of the cap.

FIG. 3A. Shows the device in the cut view separated by pieces number (6A) shows the smaller piece with its graduated cone number (6B) shows the larger piece with its larger graduated cone shaped number (11) shows the threads on the inner side of the cap.

FIG. 4 shows the device separated by pieces in a upward view with the screens attached to the Filter in number (13) and the Cap in number (16) Number (14) is the smaller for smaller self-rolled tobacco cigarettes number (15) is for larger self-rolled Cigarettes such as when using Cigar paper.

FIG. 5 Shows the device with a thick rolled tobacco cigar number (17) inserted into the larger piece, number (3)

FIG. 6 Shows the device with a thinner rolled cigarette size tobacco cigarette number (18) inserted into the smaller piece number (2) at the inner hole at number (19)

FIG. 7 Shows the device being used without any rolling paper with the tobacco, number (20) inserted into the bigger piece, number (3)

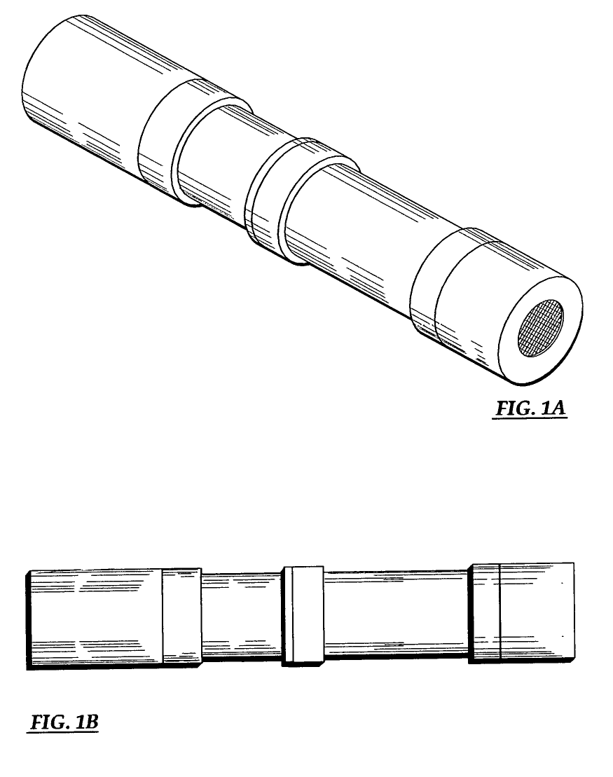

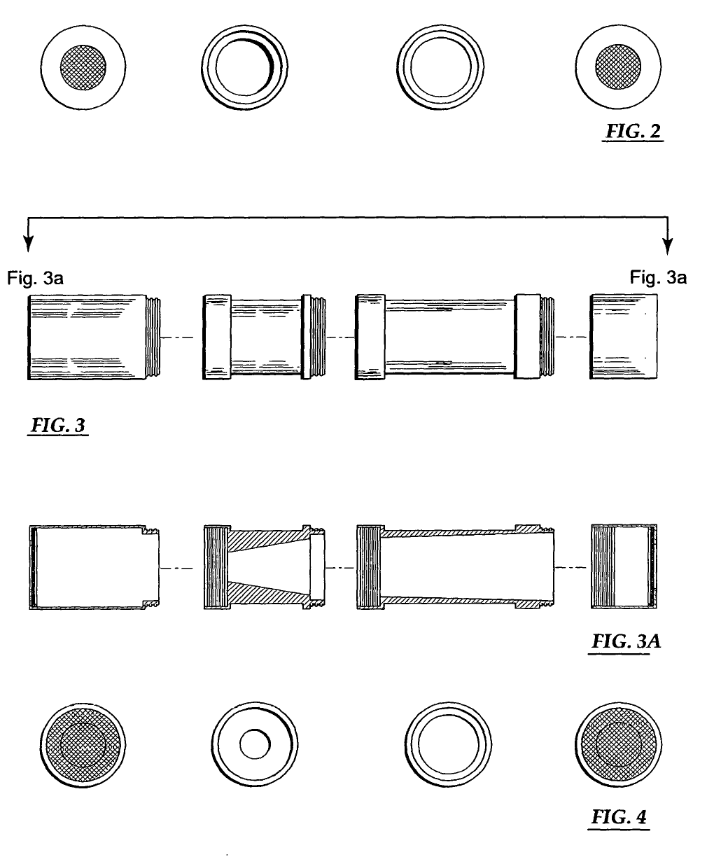

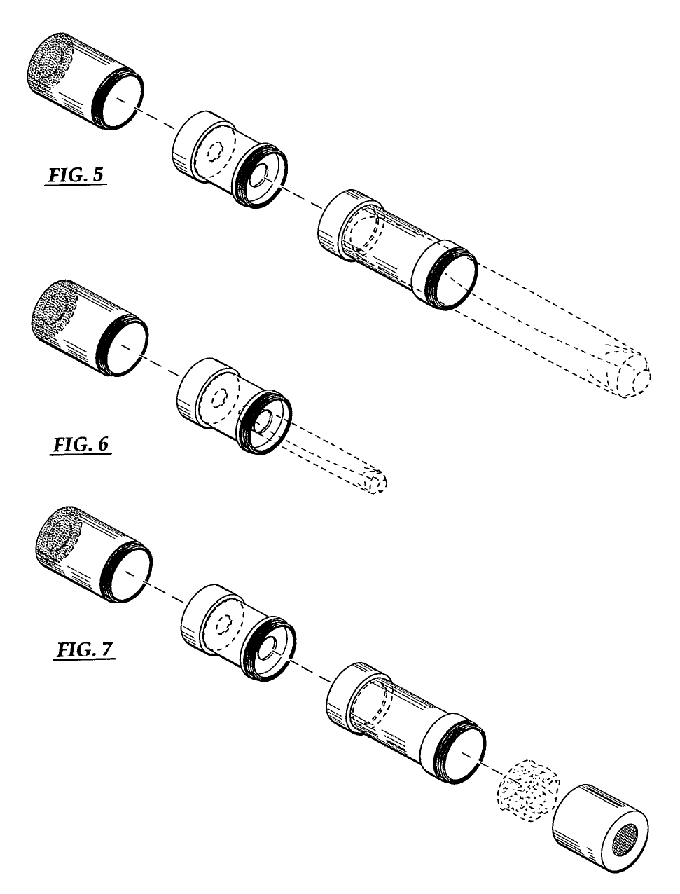

### DETAILED DESCRIPTION AND BEST MODE OF IMPLEMENTATION

Multi Purpose Self Rolled and Paperless Smoking Apparatus

It is composed of 6 parts, 4 aluminum tubes and 2 screens, in some embodiments it made be made in one or two parts. Part 1 is an aluminum filter that houses a screen that fits into a raised groove built into part 1 and it has a hole in it that allows you to inhale your tobacco whether you are using it as a filter or holder for a rolling paper or cigar paper rolled tobacco cigarette or the apparatus if you choose to use it as a paperless tobacco paper. Part 2 is an aluminum tube that has a graduated cone shaped tube that fits the size of a regular sized rolling paper and it is coned to fit various sizes and the point that is usually made when rolling a self-rolled cigarette the internal cone shape also allow it to fit a pre bought self-rolled cigarette such as the legal ones that are now allowed in cannabis legal and medical marijuana states. Part 3 is an aluminum tube that has a graduated cone shaped tube that fits the size of a larger sized rolling paper and it is coned to fit various sizes and the point that is usually made when rolling a self-rolled cigarette the internal cone shape also allow it to fit a pre bought cigars such as a swisher cigar which is then used to roll a cigarette such as the legal ones that are now allowed in cannabis legal and medical marijuana states. Part 4 is an aluminum tube smaller than the filter that also houses a screen that fits into a raised groove built into part 4 it acts as a cap that can hold in your tobacco and it has a hole in it that allows you to light the apparatus if you choose to use it as a paperless tobacco paper.

1. This Device Multi-Purpose Self Rolled or paperless Smoking Apparatus for tobacco or marijuana
Using as a holder it keeps tobacco or the like out of the users mouth with the built in filter that houses a built in screen
Can be used as a paperless cigarette
The built in filter also allows a better way to inhale more of the tobacco as more air intake acts as like a small water pipe giving a cleaner smoother inhalation and a larger inhale of the tobacco. Compared to smoking directly out of a self-rolled cigarette or cigar.
Can be used to hold left over tobacco cigarette when placing the cap on
Its internal cone shape design built into parts 2 and 3 allows for different size rolled cigarettes or the like to fit in.
